(19) United States
(12) Patent Application Publication
(10) Pub. No.: US 2008/0081995 A1
(43) Pub. Date: Apr. 3, 2008

(54) THERMAL STRAIN IMAGING OF TISSUE

(76) Inventors: Kang Kim, Ann Arbor, MI (US); Sheng-wen Huang, Ann Arbor, MI (US); Russell S. Witte, Tucson, AZ (US); Matthew O'Donnell, Seattle, WA (US)

Correspondence Address: HARNESS, DICKEY & PIERCE, P.L.C., P.O. BOX 828, BLOOMFIELD HILLS, MI, 48303, US

(21) Appl. No.: 11/866,023

(22) Filed: Oct. 2, 2007

Related U.S. Application Data
(60) Provisional application No. 60/849,083, filed on Oct. 3, 2006.

(51) Int. Cl. A61B 8/12

(52) U.S. Cl. 600/438

(57) ABSTRACT

Thermal strain imaging can be used to identify vascular plaques. Methods include heating and imaging with ultrasound to identify vulnerable plaques, which typically consist of a large lipid-rich core, in peripheral arteries of a patient. Lipid-bearing tissue has a negative temperature dependence of sound speed, whereas water-based tissue has a positive one, allowing thermal strain imaging to differentiate the two different types of tissues with high contrast to characterize plaque composition. Apparatus and methods of the present teachings allow noninvasive and reliable plaque identification.

### CROSS-REFERENCE TO RELATED APPLICATIONS

This application claims priority to U.S. Provisional Application No. 60/849,083 filed on Oct. 3, 2006, which is incorporated by reference.

All literature and similar materials cited in this disclosure, including but not limited to, patents, patent applications, articles, books, treatises, and internet web pages, regardless of the format of such literature and similar materials, are expressly incorporated by reference in their entirety for any purpose. In the event that one or more of the incorporated literature and similar materials differs from or contradicts this disclosure, including but not limited to defined terms, term usage, described techniques, or the like, this disclosure controls.

### GOVERNMENT RIGHTS

Portions of the present disclosure were made with U.S. Government support under National Institutes of Health grant EB003451. The U.S. Government has certain rights in this disclosure.

### FIELD

The present disclosure relates to apparatus and methods of inducing and imaging thermal strain to identify plaques using ultrasound, such as plaques on arterial and vascular wall tissue.

### INTRODUCTION

The statements in this section merely provide introduction information related to the present disclosure and may not constitute prior art.

Among all atherosclerotic lesions, vulnerable plaque, which usually consists of a large lipid-rich core, is particularly lethal and its sudden rupture typically leads to intraluminal thrombus, as described in Naghavi, M. et al., “From vulnerable plaque to vulnerable patient: A call for new definitions and risk assessment strategies: Part I,” Circulation, vol. 108, pp. 1664-1672, 2003. Imaging techniques such as intravascular ultrasound, ultrafast computed tomography, and magnetic resonance imaging can detect atherosclerotic plaques. Exemplary imaging techniques are disclosed in Z. A. Fayad and V. Fuster, “Clinical imaging of the high-risk or vulnerable atherosclerotic plaque,” Circulation Res., vol. 89, pp. 305-316, 2001). However, these techniques are either invasive or lack sufficient spatial and contrast resolution to reliably identify high-risk arterial plaques.

Temperature dependence of sound speed can be used for plaque composition characterization, defined by the relationship:

$$λ ≡ \frac{\partial c}{c \partial T} , \qquad ( 1 )$$

where c is the sound speed and Tis the temperature. The value of λ (° C.⁻¹) ranges from −1.3×10⁻³ to −2×10⁻³ and from 0.7×10⁻³ to 1.3×10⁻³ for lipid- and water-bearing tissues, respectively, as reported in F. A. Duck, Physical Properties of Tissue. London: Academic, 1990. Because of the sign change, thermal strain imaging (TSI) can differentiate the two different types of tissues with high contrast and thus is useful for identification of vulnerable atherosclerotic plaques, as described by Y. Shi, R. S. Witte, and M. O'Donnell, “Identification of vulnerable atherosclerotic plaque using IVUS-based thermal strain imaging,” IEEE Trans. Ultrason., Ferroelect., Freq. Contr., vol. 52, no. 5, pp. 844-850, 2005. Note that the methods reported by Shi et al. used microwave radiation to induce the thermal strain, which results from sound speed change with temperature.

Vascular disease is a significant health issue. Consequently, there is a need for a non-invasive method for detecting plaques on arterial and vascular wall tissue. Furthermore, improvements in imaging resolution and contrast would provide better detection and diagnosis of vascular pathologies, including atherosclerotic lesions such as vulnerable plaque.

### SUMMARY

In some embodiments, the present disclosure provides a method of identifying vascular plaques and their compositions. The method may include heating a vascular region of interest using ultrasound and imaging thermal strain of the region of interest using ultrasound. The imaging may differentiate between lipid- and water-bearing tissues in the vascular region of interest.

In some embodiments, the present disclosure provides a method of discriminating between fatty and water-based tissues. The method may include heating tissue with ultrasound and imaging temporal strain contrast within the heated tissue using an echo shift tracking algorithm, such as correlation-based speckle tracking. The tissue type and composition may be resolved based on the temporal strain contrast.

In some embodiments, the present disclosure provides a system for thermal strain imaging. The system may include an ultrasound heating array, an ultrasound imaging array, and a processor. The processor may be capable of processing radio-frequency image data collected by the ultrasound imaging array to estimate thermal strain produced by the ultrasound heating array.

The present teachings provide various benefits including apparatus and methods of thermal strain imaging using ultrasound heating in order to identify high-risk plaques in the major arteries such as carotids and peripheral arteries. The present teachings further provide non-invasive methods to ascertain the vascular health of a patient and aid in diagnosis of vulnerable plaque in a patient. Improvements in imaging contrast and resolution allow reliable identification of plaques.

### DRAWINGS

The drawing described herein are for illustration purposes only and are not intended to limit the scope of the present teachings in any way.

FIG. 1 is a schematic illustration of an exemplary apparatus constructed in accordance with the teachings of the present disclosure;

FIG. 2 is a thermal strain image after two seconds of heating;

FIG. 3 diagrams the relationship between an apparatus array, an artery having a region of interest, and ultrasonic intensity;

FIG. 4 graphically depicts intensity delivered into the region of interest; and

FIG. 5 (a)-(d) graphically depict the results for a 6 mm region of interest width and 40 mm elevational focus.

### DESCRIPTION

Further areas of applicability will become apparent from the description provided herein. It should be understood that the description and specific examples are intended for purposes of illustration only and are not intended to limit the scope of the present disclosure.

The present disclosure provides methods and apparatus for inducing and imaging thermal strain to identify vulnerable plaques in peripheral arteries based on ultrasound scanners. Vulnerable plaque usually consists of a large lipid-rich core. Because lipid-bearing tissue has a negative temperature dependence on sound speed, whereas water-based tissue has a positive one, thermal strain imaging can differentiate the two different types of tissues with high contrast and thus is useful for plaque composition characterization.

In some embodiments, the present teachings include inducing thermal strain with the same linear array used for imaging to provide a thermal strain imaging system that is highly compatible with conventional scanners. Also provided is a technique to design ultrasound heating patterns based on linear programming. Simulation results based on a linear array (64 elements, 5 MHz, and 0.3-mm element spacing) show that raising the temperature in a region of interest (10 mm wide) 30 mm from the array by 1.9° C. within 1 second is possible even if the tissue is highly attenuating (e.g., 0.8 dB/MHz/cm).

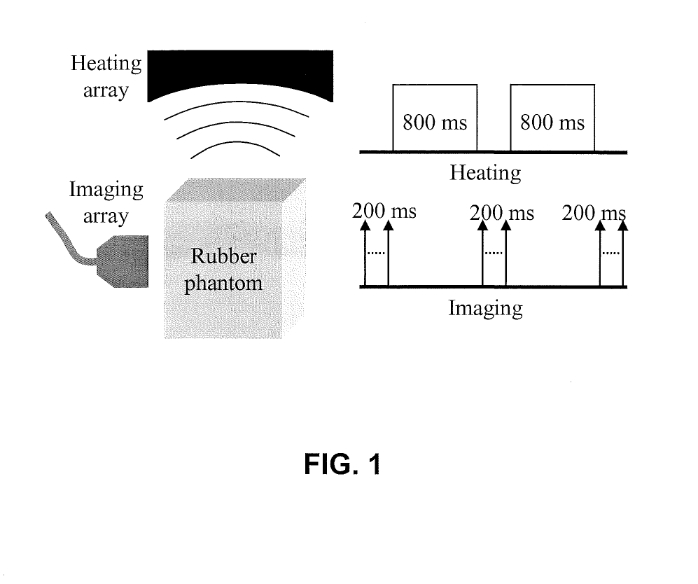

In order to confirm that within a reasonable heating time ultrasound with a limited intensity can induce thermal strains large enough for identifying tissue types, a 1-MHz 512-channel array (Imasonic, Besançon, FR), was used for heating a homogeneous rubber phantom and a commercial scanner (iU22, Philips, Bothell, Wash.) for imaging the phantom was conducted. The 1-MHz 512-channel array used is further described in T. L. Hall, J. B. Fowlkes, and C. A. Cain, “Imaging feedback of tissue liquefaction (histotripsy) in ultrasound surgery,” in Proc. IEEE Ultrason. Symp., 2005, pp. 1732-1734. The apparatus setup and the pulse sequences for heating and imaging are shown in FIG. 1.

The density ρ and the absorption coefficient α of the rubber phantom were measured to be 0.16 dB/cm/MHz and 0.87 g/cm³, respectively. The spatial average temporal average intensity I for heating over a 5 mm×5 mm region was estimated to be 74 W/cm². Therefore, the heat deposition due to absorption of ultrasound was Q=2αfI/8.69=2.7 W/cm³, where f is 1 MHz. Assuming that the specific heat C of the rubber is 2.01 J/g/° C., an estimate of the temperature rise rate based on the heat deposition is Q/ρC=1.56° C./s. The specific heat of rubber is taken from The Engineering Tool Box at http://www.engineeringtoolbox.com. Because rubber is a lipid-bearing material, a reasonable prediction of the thermal strain induced by a 2-second heating is between 0.41% and 0.62%.

A correlation-based phase-sensitive two-dimensional (2D) speckle tracking algorithm was applied to the radio-frequency image data collected by the iU22 scanner to estimate the thermal strain, which is the temporal strain along the imaging beam direction. The two-dimensional speckle tracking algorithm is based on M. A. Lubinski, S. Y. Emelianov, and M. O'Donnell, “Speckle tracking methods for ultrasonic elasticity imaging using short-time correlation,” IEEE Trans. Ultrason., Ferroelect., Freq. Contr., vol. 46, pp. 82-96, 1999. Other echo shift tracking algorithms may also be used.

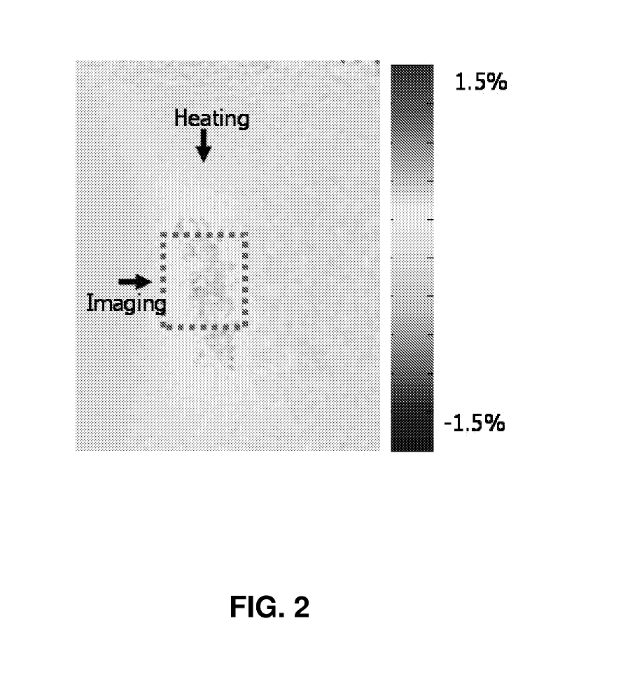

FIG. 2 shows the thermal strain image obtained after a 2-second heating. The average strain in the region indicated by the dashed box shown in FIG. 2 was 0.56%, which matches the range predicted above. The result supports that ultrasound is a suitable heating source for plaque composition characterization using TSI.

Heating Pattern Design: A thermal strain imaging system is provided that is compatible with conventional ultrasound scanners and that induces thermal strain with the same linear array used for imaging. Thus, some embodiments of the present systems integrate the heating source with the imaging system. These systems can be used with designs of ultrasound heating patterns based on linear arrays. Note that continuous ultrasound wave is used for heating. Other ultrasound wave possibilities exist for heating, including ultrasound pulses such as chirps or combinations of different ultrasound waves.

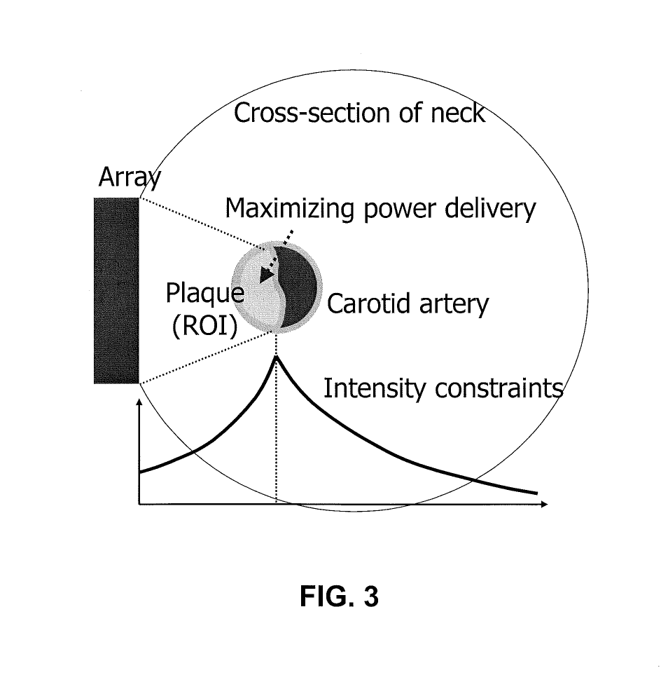

FIG. 3 illustrates the heating pattern design. To achieve enough temperature rise (1-2° C.) for plaque characterization with minimal motion artifacts, heat deposition into a plaque or a region of interest (ROI) should be maximized. On the other hand, to avoid violating spatial-peak pulse-average intensity (I_{SPPA}) limits (190 W/cm²) and overheating other tissues, ultrasonic intensities at all points should be well confined. Because attenuation coefficients in the tissues might be overestimated or underestimated, it is reasonable to allow of the maximum intensity Iₘₐₓ (≦I_{SPPA}) and lower intensities in the ROI and other regions, respectively. Specifically, the problem to be solved has the following form:
m [symbol] x x^{H}Ax subject to |Bx|≦b, (2)
where x is a complex column vector representing the weighting function (or apodization), including phase and amplitude, of the array, A is a complex positive-definite matrix related to the ROI and the radiation pattern of a single element in the array, B is a complex matrix also related to the radiation pattern of a single element, and b is a real column vector specifying the intensity constraints; defined by:

$$y = [ \begin{matrix}Re \{ x \} \\ Im \{ x \}\end{matrix} ] , \qquad ( 3 )$$

where Re{•} and Im{•} denote the real and imaginary parts, respectively. Then equation (2) can be transformed into

$${max}_{y} {\sum }_{n} {( {v}_{n}^{T} y )}^{2} subject to \hat{B} y \leq \hat{b} , \qquad ( 4 )$$

where all the matrices and vectors are real and {vₙ} is an orthogonal vector set related to A. The maximum values of equations (2) and (4) can be arbitrarily close to each other via handling \hat{B} and \hat{b}.

The problem defined in equation (4) is a concave quadratic programming problem with linear constraints and an optimal solution can be found systematically, as disclosed in R. Horst, P. M. Pardalos, and N. V. Thoai, Introduction to Global Optimization; The Netherlands: Kluwer Academic Publishers, 1995. However, for heating pattern design, the dimension of the problem is too big to be solved within a reasonable time. Therefore, equation (4) can be modified into the following problem, which is more practical in terms of time consumption:

$${max}_{y ∈ \{ {y}_{k} \}} {\sum }_{n} {( {v}_{n}^{T} y )}^{2} , {y}_{k} = arg {max}_{y} ( {v}_{k}^{T} y ) s . t . \hat{B} y \leq \hat{b} , \qquad ( 5 )$$

Each subproblem in equation (5) is a linear programming problem and solvable, as described in S. G. Nash and A. Sofer, Linear and Nonlinear Programming. New York: McGraw-Hill, 1996.

Ultrasound heating patterns can be designed based on equation (5) using MATLAB (Mathworks, Natick, Mass.) together with a free linear programming solver Ip_solve, as provided by [http://Ipsolve.sourceforge.net/5.5/]. The array was assumed to have 64 elements, an element width of 0.25 mm, an element spacing of 0.3 mm, and an element height of 8.1 mm. The frequency for heating was 5 MHz, and both the absorption and attenuation coefficients were assumed to be 0.3 dB/cm/MHz. The ROIs were parallel to and 30 mm from the array, and on the imaging plane.

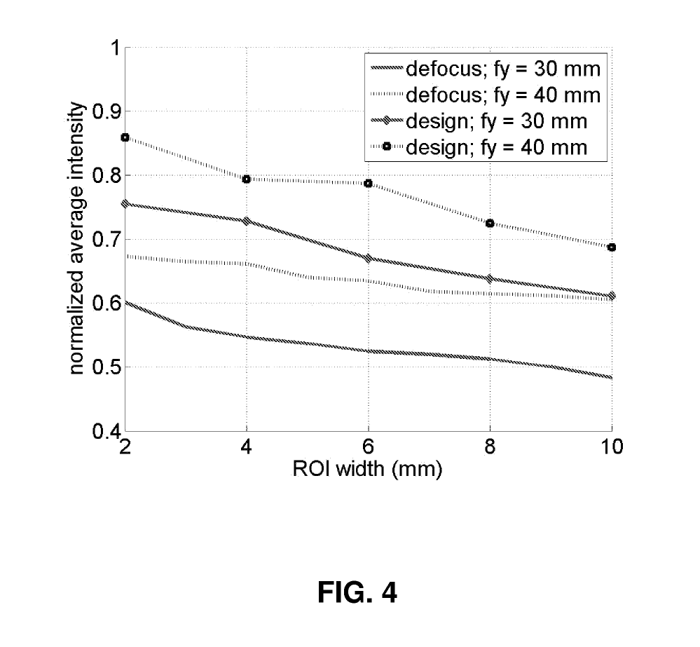

FIG. 4 shows the average intensities (normalized with respect to the maximum allowable intensity Iₘₐₓ) delivered into the ROIs with different widths. Two elevational foci, 30 mm and 40 mm, were considered, and the corresponding results are shown as a solid line with diamonds and a black dotted line with squares, respectively. Also shown in FIG. 4 are the results obtained by defocusing, which means choosing a focus deeper than the ROI position so that the intensity delivery could be increased without violating the intensity constraints. Note that in the defocusing method the optimal combination of the focus and the number of elements was found for each ROI width. Also note that all the on elements were equally weighted. The use of the proposed method can enhance the intensity delivery and hence the temperature rise rate by over 13% compared to the defocusing method. Assuming that thermal diffusion can be ignored, then the designed heating pattern can provide a temperature rise rate of 10.7° C./s if Iₘₐₓ=190 W/cm², the elevational focus is 40 mm, the ROI width is 10 mm, and the tissue density and specific heat are 1 g/cm³ and 4.2 J/g/° C., respectively. Even if the attenuation and absorption coefficients are 0.8 and 0.3 dB/cm/MHz, respectively, the temperature rise rate is still 1.9° C./s.

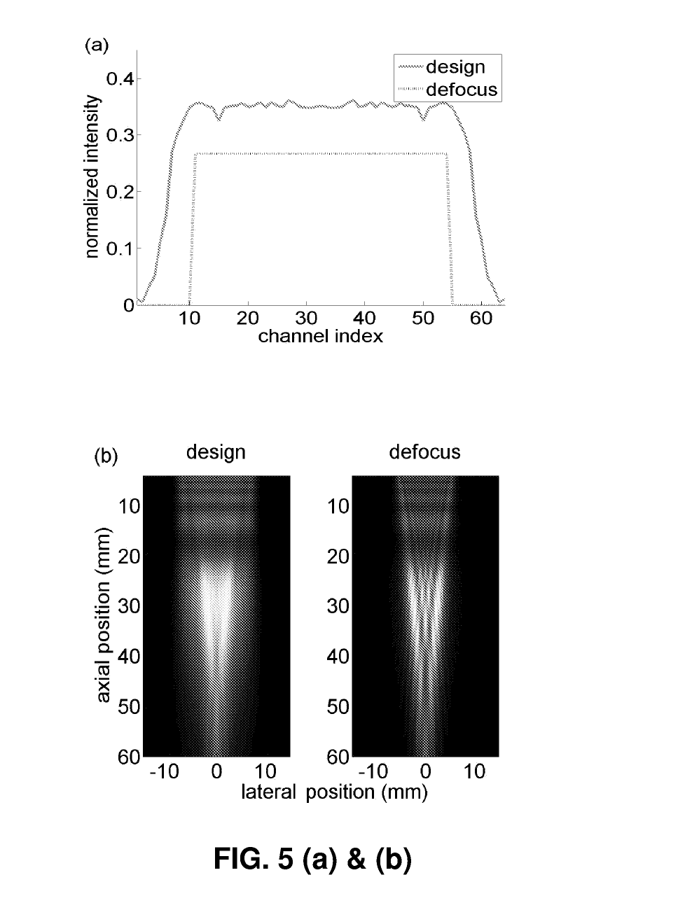

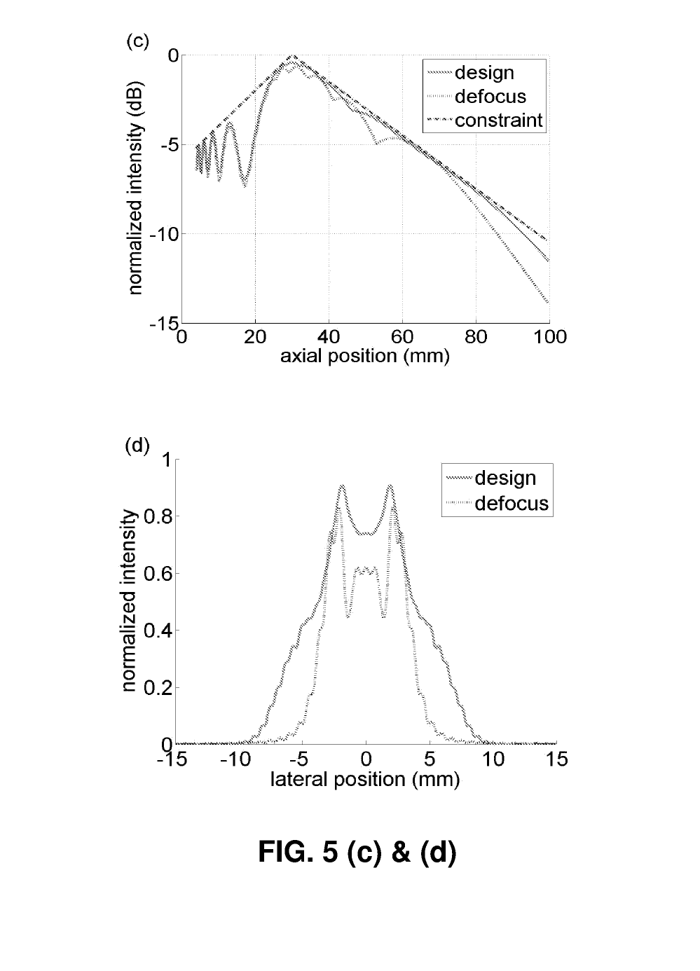

The results for the case of 6-mm ROI width and 40-mm elevational focus are shown in FIG. 5, as an example. The intensity profiles on the array surface are plotted in FIG. 5(a). For the defocusing method, the optimal combination was 44 elements and 94 mm focus. FIG. 5(b) shows the normalized intensity patterns on the imaging plane in linear scale. The maximum intensities at different depths are shown in FIG. 5(c) together with the applied constraints. Note that the maximum intensity did not necessarily locate on the axial axis. The constraint curve had a slope of 0.4 (−0.3) dB/cm/MHz before (after) the ROI. Therefore, even if the tissue before (after) the ROI has an attenuation coefficient of 0.7 (0) dB/cm/MHz, the intensity in the ROI is still close to the maximum intensity in the whole region. That is, overheating tissues other than that in the ROI can be avoided even if the attenuation coefficient distribution is out of expectation but in a reasonable range. FIG. 5(d) shows the intensity distributions at the ROI depth.

Note that heat conduction due to blood flow was not taken into account. However, finite-element analysis showed that, if the heating time is 1 second, temperature rises will be affected for 5% only in regions close to lumen (within 1 mm). Therefore, the effects of blood flow are negligible for plaques not too small or not too close to lumen.

The present teachings provide methods to design ultrasound heating patterns based on linear programming. Other methods to design ultrasound heating patterns are also possible; these methods may be based on linear programming, quadratic programming, genetic programming, and combinations thereof. Simulation results based on a linear array (64 elements, 5 MHz, and 0.3-mm element spacing) show that the use of the proposed technique can enhance the intensity delivery and hence the temperature rise rate by over 13% compared to the defocusing method. Assuming that thermal diffusion can be ignored, the designed heating pattern can provide a temperature rise rate of 10.7° C./s for a region of interest (ROI) (of 10 mm width) 30 mm from the array without violating I_{SPPA} limits. Even if the tissue is highly attenuating (0.8 dB/cm/MHz attenuation coefficient), raising the temperature by 1.9° C. within 1 second is possible. The present teachings include methods using a 1-MHz 512-channel array for heating a rubber phantom and a commercial scanner for imaging the phantom. The temperature rise rate in the heated region was estimated to be over 1° C./s using an average intensity of 74 W/cm². Therefore, thermal strain imaging using ultrasound heating can identify high-risk plaques in peripheral arteries. Furthermore, according to simulation results disclosed herein, a conventional ultrasound scanner can be modified into a TSI system if its array can maintain intensities producing I_{SPPA} limits in the ROI for a period of one second.

### EXAMPLE A

### Rabbit Kidney Model

It is well known that lipids have a negative temperature dependence of the sound speed, whereas water-based tissues have positive temperature dependence [F. A. Duck, Physical Properties of Tissue. London: Academic, 1990]. Controlled local temperature modulation can be used to image the spatial distribution of temporal strain produced by changes in the sound speed [T. Bowen, “Radiation-induced Thermoacoustic Soft-Tissue Imaging,” IEEE Transactions on Sonics and Ultrasonics, vol. 29, pp. 187-187, 1982]. The opposite sign of the two different tissue types creates the contrast required for resolving the fatty tissue from surrounding water-based tissue.

Using a 2-D phased array in combination with a conventional ultrasound scanner, the feasibility of ultrasound inducing and imaging of thermal strain is demonstrated. Among other heating sources, including microwave [Y. Shi, R. S. Witte, and M. O'Donnell , Identification of Vulnerable Plaque Atherosclerotic Plaque Using an IVUS-Based Thermal Strain Imaging, IEEE Transactions on Ultrasonics, Ferroelectrics, and Frequency Control, Vol. 52, No. 5, (May 2005)], ultrasound may be the best candidate in terms of delivering energy locally and simplicity of probe design for an existing conventional ultrasound scanner. Thermal lens effects and radiation force generated by the heating transducer, combined with motion artifacts, are issues to overcome for clinical applications. High resolution TSI using a conventional US scanner (iU22, Philips) was first applied to a uniform rubber (gel) phantom. 1-MHz 512-channel transducer array (Imasonic, Besançon, FRANCE) was used as a heating source. 2-D phase-sensitive, correlation-based speckle tracking was applied to map the spatial distribution of temporal strain across the sample.

The temperature rise in a rubber (gelatin) phantom was estimated to be 2° C. (1° C.) within 1 second using an average intensity of 100 W/cm². The thermal lens effect was also imaged by tracking the lateral displacement. To avoid radiation force, if any, the image frames before and after heating were averaged for a period of time. A rabbit kidney was prepared in a clear gel phantom. The heating beam was designed to cover 10×10×20 mm region. The heating of 800 ms was interleaved with the imaging of 200 ms. With the same intensity used above for 3 sec, the temperature was increased by about 1° C. The fatty tissue surrounding the collecting system was clearly differentiated and 2-D TSI matches well with the anatomy.

These in-vitro results demonstrate the feasibility of high resolution US induced TSI with a small temperature change over a short period of time.

The description of the technology is merely exemplary in nature and, thus, variations that do not depart from the gist of the present disclosure are intended to be within the scope of the invention. Such variations are not to be regarded as a departure from the spirit and scope of the invention.

What is claimed is:

1. A method of identifying vascular plaques comprising:
heating a vascular region of interest using ultrasound; and
imaging thermal strain of the region of interest using ultrasound, wherein the imaging differentiates between lipid- and water-bearing tissues in the vascular region of interest.

2. The method of identifying vascular plaques according to claim 1, wherein the heating step and the imaging step are performed with different ultrasound arrays.

3. The method of identifying vascular plaques according to claim 1, wherein the heating step and the imaging step are performed with the same ultrasound array.

4. The method of identifying vascular plaques according to claim 1, wherein heating a vascular region of interest using ultrasound includes using a heating pattern based on linear programming, quadratic programming, genetic programming, and combinations thereof.

5. The method of identifying vascular plaques according to claim 1, wherein heating a vascular region of interest using ultrasound raises the temperature in a region of interest from about 1° C./sec to about 10.7° C./sec.

6. The method of identifying vascular plaques according to claim 1, wherein heating a vascular region of interest using ultrasound includes using a continuous ultrasound wave or chirps.

7. The method of identifying vascular plaques according to claim 1, wherein imaging thermal strain of the region of interest using ultrasound includes processing radio-frequency image data to estimate thermal strain produced by the heating step.

8. The method of identifying vascular plaques according to claim 7, wherein processing radio-frequency image data to estimate thermal strain produced by the heating step includes an echo shift tracking algorithm.

9. The method of identifying vascular plaques according to claim 8, wherein the echo shift tracking algorithm is a correlation-based, phase-sensitive, two-dimensional speckle tracking algorithm.

10. A method of discriminating between fatty- and water-based tissues comprising:
heating tissue with ultrasound;
imaging temporal strain contrast within the heated tissue using an echo shift tracking algorithm; and
resolving the tissue type based on the temporal strain contrast.

11. The method of discriminating between fatty- and water-based tissues according to claim 10, wherein the echo shift tracking algorithm in the imaging step is correlation-based speckle tracking.

12. The method of discriminating between fatty- and water-based tissues according to claim 10, wherein the heating step and the imaging step are performed with the same ultrasound array.

13. The method of discriminating between fatty- and water-based tissues according to claim 10, wherein heating tissue with ultrasound includes using a heating pattern based on linear programming, quadratic programming, genetic programming, and combinations thereof.

14. The method of discriminating between fatty- and water-based tissues according to claim 10, wherein heating tissue with ultrasound raises the temperature in a region of interest from about 1° C./sec to 10.7° C./sec.

15. The method of discriminating between fatty- and water-based tissues according to claim 10, wherein heating tissue with ultrasound includes using a continuous ultrasound wave or chirps.

16. A system for thermal strain imaging comprising:
an ultrasound heating array;
an ultrasound imaging array;
a processor capable of processing radio-frequency image data collected by the ultrasound imaging array to estimate thermal strain produced by the ultrasound heating array.

17. The system for thermal strain imaging according to claim 16, wherein the processor includes an echo shift tracking algorithm.

18. The system for thermal strain imaging according to claim 16, wherein the echo shift tracking algorithm is a correlation-based, phase-sensitive, two-dimensional speckle tracking algorithm.

19. The system for thermal strain imaging according to claim 16, wherein the ultrasound heating array is separate from the ultrasound imaging array.

20. The system for thermal strain imaging according to claim 16, wherein the ultrasound heating array and the ultrasound imaging array are integrated.

21. The system for thermal strain imaging according to claim 16, wherein the ultrasound heating array and the ultrasound imaging array are the same ultrasound array.

22. The system for thermal strain imaging according to claim 16, wherein the ultrasound heating array is capable of producing a continuous ultrasound wave or chirps.
